(19) United States
(12) Patent Application Publication
(10) Pub. No.: US 2026/0207243 A1
(43) Pub. Date: Jul. 23, 2026

(54) Catheter for Treating Calcified Plaque

(71) Applicant: Medtronic Vascular, Inc, Santa Rosa, CA (US)

(72) Inventors: Kelsey M. Sandquist, Santa Rosa, CA (US); Enda F. Carter, Multyfarnham (IE); David Killeen, Glenanail (IE); Laura M. O'Shea, Tralee (IE); Darion R. Peterson, Longmont, CO (US); Gemma Lynch, Lifford (IE); Simon Coyle, Ballinasloe (IE); Binit Panda, Santa Rosa, CA (US); Tanay Garg, Petaluma, CA (US); Cian Walsh, Oranmore (IE); Aram Jamous, Athenry (IE); Aiswarya Balakrishnan, Galway (IE); Manoj Kumar Singh, Santa Rosa, CA (US)

(21) Appl. No.: 19/136,051

(22) PCT Filed: Dec. 5, 2023
(86) PCT No.: PCT/US2023/082551

§ 371 Date: Jun. 5, 2025

Related U.S. Application Data
(60) Provisional application No. 63/386,118, filed on Dec. 5, 2022.

(51) Int. Cl. A61B 18/02; A61B 18/00; A61M 25/10

(52) U.S. Cl. CPC A61B 18/02; A61M 25/104; A61B 2018/00095; A61B 2018/0022; A61B 2018/00642; A61B 2018/00714; A61B 2018/00791; A61B 2018/0212; A61B 2018/0262; A61M 2025/1088; A61M 2205/3327; A61M 2205/3368; A61M 2205/3606; A61M 2205/50

(57) ABSTRACT

A catheter for treating calcified plaque includes a catheter body and an expandable balloon. A treatment device of the catheter disrupts and/or modifies the calcified plaque. The treatment device may be a thermal shock generator, a nuclear magnetic resonance generator, or a vibration generator.

### FIELD

The present technology is generally related to a catheter and method for treating calcified plaque within a body of a subject.

### BACKGROUND

A variety of techniques and instruments have been developed to percutaneously treat calcified plaque within a body of a subject. As an example, calcified plaque may build up within the circulatory system of the subject. A common example is the buildup of fatty deposits (atheromas) in the intimal layer (under the endothelium of a patient's blood vessels). Over time, what is initially deposited as relatively soft, cholesterol-rich atheromatous material often hardens into a calcified atherosclerotic plaque. The atheromas may be referred to as stenotic lesions or stenoses while the blocking material may be referred to as stenotic material. If left untreated, such stenoses can so sufficiently reduce perfusion that angina, hypertension, myocardial infarction, strokes and the like may result. Angioplasty or atherectomy may be performed to improve blood flow. However, the presence of calcified plaque typically leads to difficulty in adequately treating the blood vessel.

### SUMMARY

The techniques of this disclosure generally relate to modifying and/or disrupting calcified plaque.

In one aspect, the present disclosure provides catheter for treating calcified plaque within a body of a subject. The catheter comprises a catheter body having opposite proximal and distal end portions and a longitudinal axis extending therebetween. The catheter body is configured to be percutaneously inserted into the body of the subject and delivered to a treatment site having calcified plaque. An expandable balloon is coupled to the distal end portion of the catheter body. The expandable is configured to contact the calcified plaque and apply a radial pressure thereto. Aa thermal shock generator is operatively coupled to the expandable balloon and configured to alternate between heating and cooling the calcified plaque to induce thermal shock in the calcified plaque.

In another aspect, the disclosure provides a method of treating calcified plaque at a treatment site within a body of a subject. The method comprises delivering a catheter body of a catheter to the treatment site so that a balloon at a distal end portion of the catheter body is adjacent the calcified plaque; expanding the balloon after said delivering the catheter body to apply radial pressure to the calcified plaque; heating the calcified plaque; and rapidly cooling the heated calcified plaque to induce thermal shock in the calcified plaque simultaneously with the radial pressure applied to the calcified plaque by the expandable balloon.

In yet another aspect, the disclosure provides a catheter for treating calcified plaque within a body of a subject. The catheter comprises a catheter body having opposite proximal and distal end portions and a longitudinal axis extending therebetween. The catheter body is configured to be percutaneously inserted into the body of the subject and delivered to a treatment site having calcified plaque. A nuclear magnetic resonance generator includes a radiofrequency coil coupled to the distal end portion of the catheter body. The nuclear magnetic resonance generator is configured to disrupt the calcified plaque.

In still another aspect, the disclosure provides a catheter for treating calcified plaque within a body of a subject. The catheter comprises a catheter body having opposite proximal and distal end portions and a longitudinal axis extending therebetween. The catheter body is configured to be percutaneously inserted into the body of the subject and delivered to a treatment site having calcified plaque. A vibration generator at the distal end portion of the catheter body is configured to generate radial mechanical vibrations suitable to produce resonance in calcified deposits in the calcified plaque, thereby disrupting the calcified deposits.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic of an embodiment of a catheter for treating calcified plaque.

FIG. 2 is schematic cross section of a catheter body of the catheter.

FIG. 3 is a schematic of the catheter in a body lumen including calcified plaque, a balloon of the catheter in a deflated state.

FIG. 4 is similar to FIG. 3 with the balloon in an inflated state and receiving heated fluid therein to heat the calcified plaque.

FIG. 5 is similar to FIG. 4 except with cooled fluid being delivered to the balloon to rapidly cool the calcified plaque.

FIG. 6 is an exemplary protocol for a control unit operating the catheter.

FIG. 7 is another embodiment of a catheter for treating calcified plaque using thermal shock treatment.

FIG. 8 is yet another embodiment of a catheter for treating calcified plaque using nuclear magnetic resonance.

FIG. 9 is another embodiment of a catheter similar to FIG. 8.

FIG. 10 is another embodiment of a catheter for treating calcified plaque using nuclear magnetic resonance.

FIG. 11 is embodiment of a catheter for treating calcified plaque using mechanical vibrations.

FIG. 12 is an enlarged distal end portion of the catheter in FIG. 11.

FIG. 13 is another embodiment of a catheter treating calcified plaque using mechanical vibrations.

FIG. 14 is an enlarged distal end portion of the catheter in FIG. 13.

FIG. 15 is an enlarged distal end portion of another embodiment of a catheter treating calcified plaque using mechanical vibrations.

FIG. 16 is an enlarged distal end portion of yet another embodiment of a catheter treating calcified plaque using mechanical vibrations.

### DETAILED DESCRIPTION

The following description is generally related to embodiments and examples of a treatment catheter for treating calcified plaque within a body of a subject. The illustrated examples are suitable for treating calcified plaque within a circulatory system of the subject, such as blood vessels and/or the heart of the subject. The illustrated examples may also be suitable for treating other body lumen outside the circulatory system.

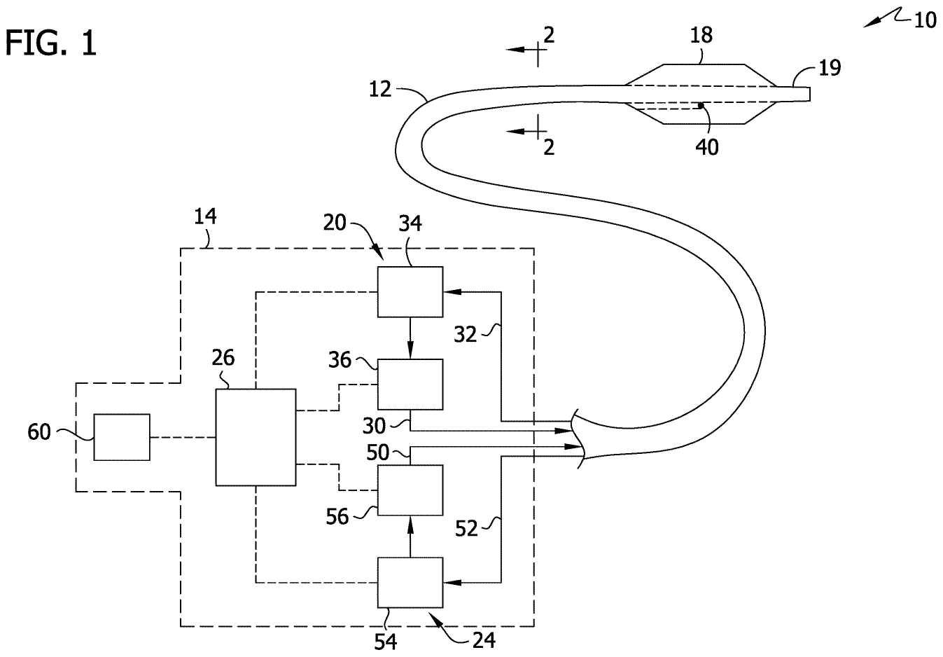

Referring to FIG. 1, one embodiment of a treatment catheter for treating calcified plaque within a body of a subject is generally indicated at reference numeral 10. In general, the catheter 10 is configured to create thermal shock in the calcified plaque.

The thermal shock modifies or disrupts the calcified plaque. For example, the thermal shock may fracture the calcified plaque, thereby facilitating treatment. In one example, the calcified plaque may be treated further, such as through angioplasty or atherectomy or other treatments, or the treatment using thermal shock may be the primary or only treatment of the calcified plaque.

Referring still to FIG. 1, the catheter 10 includes a catheter body 12 having proximal and distal end portions and a longitudinal axis extending therebetween. The catheter body 12 is designed and constructed to be percutaneously inserted into a blood vessel of the subject to deliver the distal end portion of the catheter body to the treatment site including calcified plaque. As non-limiting examples, the catheter body 12 may have a length from about 135 cm to about 142 cm, and a diameter from about 138 mm to about 142 mm. The catheter body 12 may suitably comprise a flexible material, such as a polymer, to enable the body to traverse a tortuous path to the treatment site.

A thermal shock generator 14 of the catheter 10 is configured to heat (i.e., transfer heat to) and then rapidly cool (i.e., remove heat from) the calcified plaque to create thermal shock within the calcified plaque. In the present embodiment, the thermal shock generator 14 is fluidly connected to an expandable heat transfer element 18 at the distal end portion of the catheter body. The thermal shock wave generator 14 includes a heating system, generally indicated at 20, in thermal communication with the heat transfer element, a cooling system, generally indicated at 24, in thermal communication with the heat transfer element, and a control unit 26 controlling operating of the heating and cooling systems. As explained in more detail below, the heating system 20 is configured to generate and transfer heat to the heat transfer element, which in turn transfers heat to the calcified plaque at the treatment site. The cooling system 24 is configured to rapidly remove heat from the transfer element, which is turn rapidly removes heat from the calcified plaque at the treatment site. In other words, the cooling system 24 is configured rapidly cool the calcified plaque. The control unit 26 is configured to control the timing and the amount of heating and cooling of the calcified plaque to create thermal shock within the plaque, such a represented in FIG. 6.

In the present illustrated embodiment, the expandable heat transfer element 18 is an expandable balloon configured to receive a thermally conductive fluid to inflate the balloon. The inflated balloon 18 contacts the calcified plaque and applies a radial pressure or force thereto. The inflated balloon 18 also facilitates heat transfer between the plaque and the inflated balloon. A wall of the balloon 18 is thermally conductive to facilitate heat transfer from the thermally conductive fluid to the wall of the balloon and from the wall of the balloon to the calcified plaque. Suitable fluid for the balloon 18 includes, but is not limited to, saline. Suitable balloon material includes, but is not limited to, Nylon. Also in the illustrated embodiment, a guidewire lumen 19 extends along the catheter body 12 and through the balloon 18 for receiving a suitable guidewire (not shown).

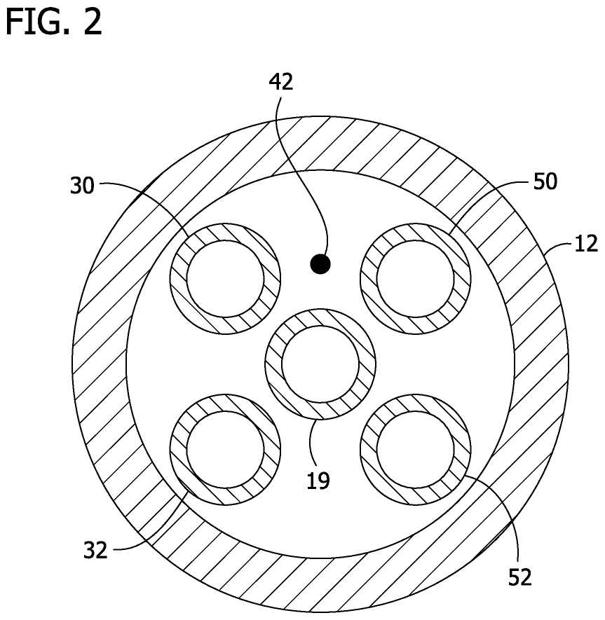

Referring to FIGS. 1 and 2, in the present illustrated embodiment, the heating system 20 includes a heating fluid lumen 30 extending along the catheter body 12 from the proximal end portion to the interior of the balloon 18, a return heating lumen 32 extending along the catheter body from the interior of the balloon toward the proximal end portion, a fluid heater 34, and a fluid circulator 36 configured to circulate the heated fluid between the fluid heater and the heating fluid lumen and return lumen. The fluid heater 34 may be a conventional heater for heating fluid, such as by conduction or in other ways. The fluid circulator 36 is configured to deliver the heated fluid through the heating fluid lumen 30 and into the balloon 18, whereupon the balloon the calcified plaque is heated through conduction, for example. The fluid is recirculated back through the return lumen 32 to be reheated and delivered to the balloon 18. The temperature of the fluid when it enters the balloon 18 may be from about 150° C. to about 160° C. As explained below, a temperature sensor 40 (e.g., thermocouple) may be disposed in, on, or otherwise in thermal communication with the balloon. The temperature sensor 40 is in communication (e.g., wired or wireless) with the control unit 26 to monitor to the temperature of the fluid. In the illustrated embodiment, a conductive wire 42 (FIG. 2) connects the temperature sensor 40 to the control unit 26.

In the present illustrated embodiment, the cooling system 24 includes a cooling fluid lumen 50 extending along the catheter body 12 from the proximal end portion to the interior of the balloon 18, a return cooling lumen 52 extending along the catheter body from the interior of the balloon toward the proximal end portion, a fluid chiller 54 disposed outside the patient's body, and a fluid circulator 56 configured to circulate the cooled fluid between the fluid chiller and the cooling fluid lumen and return lumen. The fluid chiller 50 may be a conventional chiller for chilling fluid, such as by conduction or in other ways. The fluid circulator 56 is configured to deliver the cooled fluid through the cooling fluid lumen 50 and into the balloon 18, whereupon the calcified plaque is rapidly through conduction, for example. The fluid is recirculated back through the return lumen 52 to be re-cooled and delivered to the balloon 18. The temperature of the fluid when it enters the balloon 18 may be from about −20° C. to about −40° C. As explained below, the temperature sensor 40 in communication (e.g., wired or wireless) with the control unit 26 is used monitor to the temperature of the fluid. As explained below, the calcified plaque may be rapidly cooled in other ways.

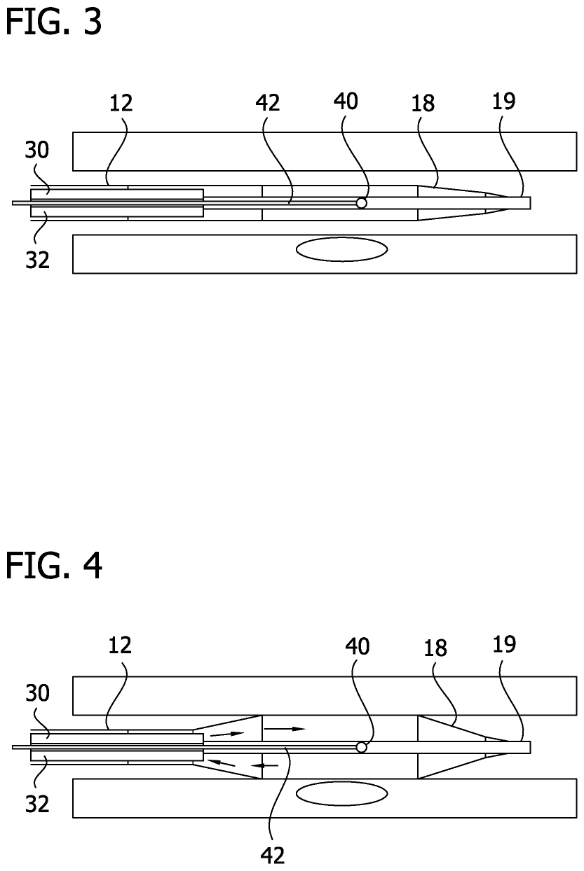

Referring to FIG. 3, in one example, the distal end of the catheter body is delivered to the treatment site such that the balloon 18, in its deflated state, is adjacent the calcified plaque. The user may then interface with the control unit 26 via a user interface 60 (e.g., touchscreen) so that the control unit actuates a treatment protocol. In one example, as shown in FIG. 4, the control unit activates the heating system 20 by activating the heater and the circulator to both inflate the balloon and heat the balloon. The temperature within or of the balloon or the temperature of the calcified plaque is monitored by the control unit through feedback from the temperature sensor 40. In one example, the balloon wall is heated to a temperature from about 150° C. to about 300° C.

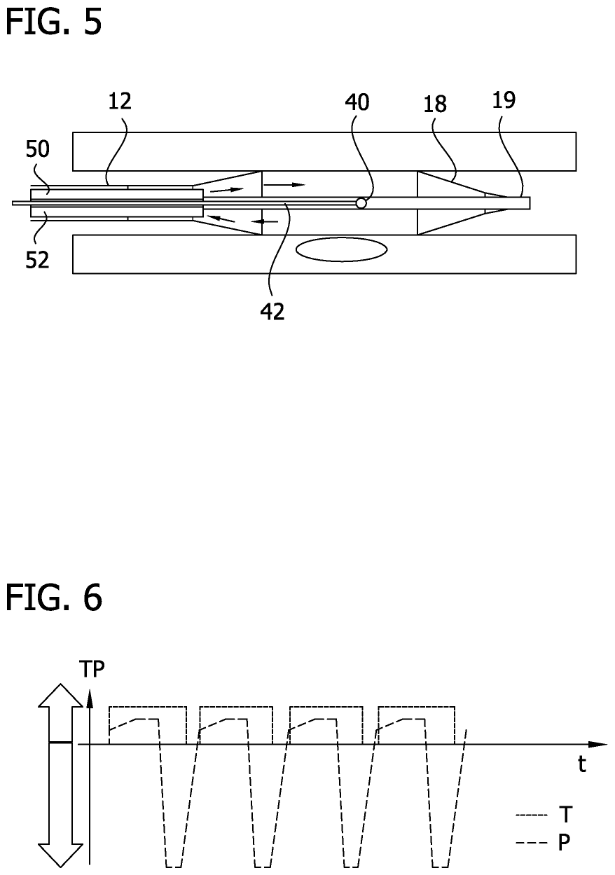

Upon reaching a heated threshold temperature signal from the temperature sensor 40 that is indicative of the balloon wall reaching a desired temperature for a desired amount of time, the control unit 26 actuates delivery of the cooled fluid to rapidly cool (or remove heat from) the balloon 18 and the calcified plaque, as shown in FIG. 5. The cooled fluid may have been pre-cooled before the control unit 26 operates the circulator 56 so that the cooled fluid immediately replaces the heated fluid to impart rapid cooling. In one example, the balloon wall is cooled at a rate from about −20° C./s to about −40° C./s. The control unit 26 may cease circulation of the cooled fluid upon reaching a threshold cooled temperature signal from the temperature sensor 40 for a desired amount of time. In one example, the threshold cooled temperature signal may be indicative of the balloon wall reaching a temperature from about −38° C. to about −40° C.

In one example, the control unit 26 may be programmed to operate the heating and cooling systems 20, 24 to perform repetitive heating and cooling of the calcified plaque and repetitive application of radial force from the balloon 18. One example of a suitable protocol for is shown in FIG. 6, with the solid line indicated temperature and the dashed line indicating pressure exerted by the balloon. The heating and subsequent rapid cooling of the calcified plaque along with the pulsed radial pressure causes fractures (e.g., stress fractures) within the calcified plaque and the inflated balloon imparts radial stress to the plaque. This combination of treatment modifies or disrupts the plaque. After treatment with the catheter 10, in one example the catheter body 12 may be withdrawn and subsequent treatment (e.g., angioplasty and/or atherectomy and/or drug treatment) may be performed.

It is understood that the operation of the heating and cooling systems 20, 24 may be reversed, so that the cooling system is activated and then subsequently the heating system is activated.

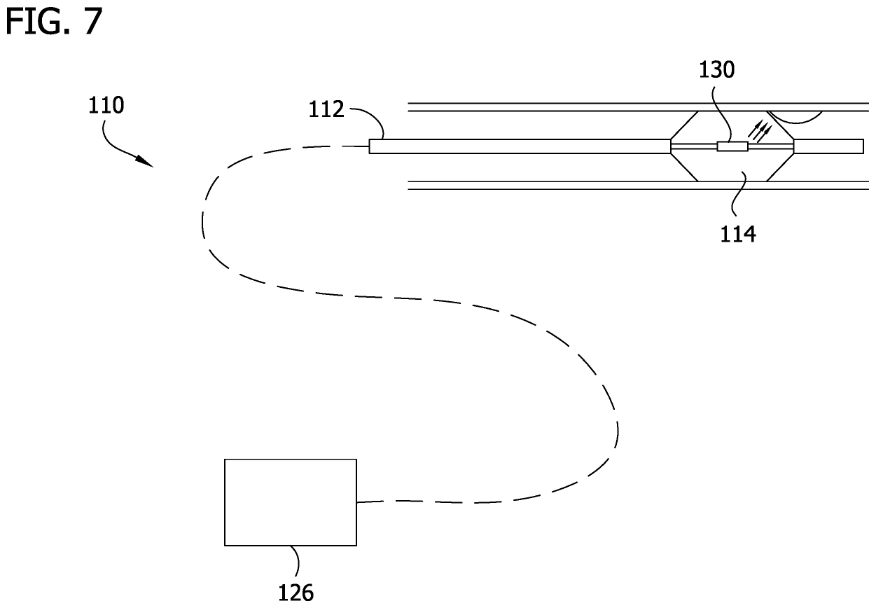

Referring to FIG. 7, another embodiment of a treatment catheter for treating calcified plaque within a body of a subject is generally indicated at reference numeral 110. In general, the catheter 110 is similar to catheter 10 in that the present catheter is configured to create thermal shock in the calcified plaque. The thermal shock modifies or disrupts the calcified plaque. For example, the thermal shock may fracture the calcified plaque, thereby facilitating treatment. In one example, the calcified plaque may be treated further, such as through angioplasty or atherectomy or other treatments, or the treatment using thermal shock may be the primary or only treatment of the calcified plaque.

In this embodiment, the catheter 110 includes a catheter body 112 and a cryoballoon 114 at a distal end portion thereof configured to rapidly cool the calcified plaque. As is generally known in the art, the cryoballoon 114 includes a refrigerant released in the balloon to rapidly cool the inflation fluid in the balloon. The cryoballoon 114 is in contact with the calcified plaque to rapidly cool the plaque. In general, the cryoballoon 114 includes a cooling system in which the fluid in the balloon is cooled in the balloon rather than the fluid being cooled remote from the balloon and then delivered to the balloon. The catheter 110 includes a control unit 126 for controlling cooling of the calcified plaque using the cryoballoon 114.

The illustrated embodiment also includes a plaque heating element 130 in or adjacent the balloon for non-conductive heating of the calcified plaque. In general, the plaque heating element 130 is part of a heating system of the catheter 110. The plaque heating element 130 may be an ultrasonic transducer for generating ultrasonic energy directed toward the calcified plaque. The ultrasonic energy is absorbed by the calcified plaque to heat the plaque. In another embodiment, the plaque heating element 130 may be a radiofrequency generator configured to heat the calcified plaque by dielectric heating. The plaque heating element 130 may be of other types for non-conductive heating. The control unit 136 is in communication with the plaque heating element 130 to operate the heating element.

In one example, the control unit 126 may be programmed to operate the cryoballoon 114 and the heating element to perform repetitive heating and cooling of the calcified plaque and repetitive application of radial force from the balloon 118. One example of a suitable protocol for is shown in FIG. 6, with the solid line indicated temperature and the dashed line indicating pressure exerted by the balloon. The heating and subsequent rapid cooling of the calcified plaque along with the pulsed radial pressure causes fractures (e.g., stress fractures) within the calcified plaque and the inflated balloon imparts radial stress to the plaque. This combination of treatment modifies or disrupts the plaque. After treatment with the catheter 110, in one example the catheter body 112 may be withdrawn and subsequent treatment (e.g., angioplasty and/or atherectomy and/or drug treatment) may be performed.

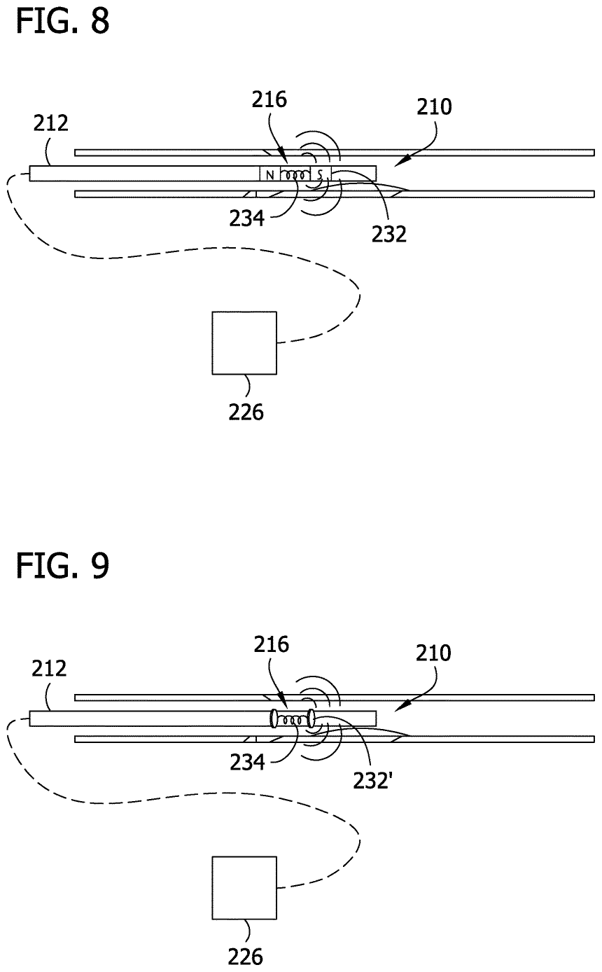

Referring to FIG. 8, another embodiment of a treatment catheter for treating calcified plaque within a body of a subject is generally indicated at reference numeral 210. In general, the catheter 210 is configured to modify or disrupt the calcified plaque using nuclear magnetic resonance (NMR). In one example, the calcified plaque may be treated further, such as through angioplasty or atherectomy or other treatments, or the treatment catheter 210 may be the primary or only treatment of the calcified plaque.

The catheter 210 includes a catheter body 212, an NMR generator, generally indicated at 216, coupled to a distal end portion of the catheter body, and a control unit 226 in communication with the NMR generator. The catheter body 212 is designed and constructed to be percutaneously inserted into a blood vessel of the subject to deliver the distal end portion of the catheter body to the treatment site including calcified plaque. As non-limiting examples, the catheter body 212 may have a length from about 132 cm to about 142, and a diameter from about 17 mm to about 20 mm. The catheter body 212 may suitably comprise a flexible material, such as plastic, to enable the body to traverse a tortuous path to the treatment site.

The illustrated NMR generator 216 includes at least one magnet 232 (broadly, a constant magnetic field generator) and at least one radiofrequency (RF) coil 234 (broadly, an oscillating magnetic field generator) adjacent the magnet. The magnet 232 produces a magnetic field that polarizes molecules in the calcified plaque. The magnet 232 may be a permanent magnet, as shown in FIG. 8, or an electromagnet 232′, as shown in FIG. 9. The RF coil 234 produces oscillating magnetic field at the Larmor frequency of calcium to “relax” the molecules. This relaxation of the molecules disturbs the calcium in the calcified plaque. The control unit 226 controls operation of the RF coil to produce the Larmor frequency.

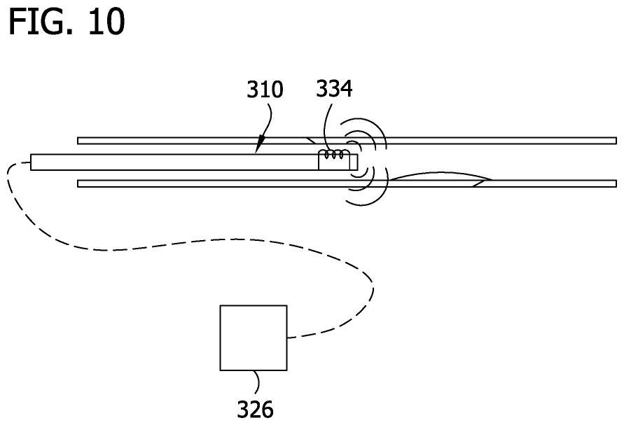

Referring to FIG. 10, in yet another embodiment similar to the catheter 210, a catheter 310 includes an RF coil 334 (broadly, an oscillating magnetic field generator) controlled by control unit 326 but does not include a magnet. Instead, the contact magnetic field is generated outside the subject's body, such as by an MRI machine.

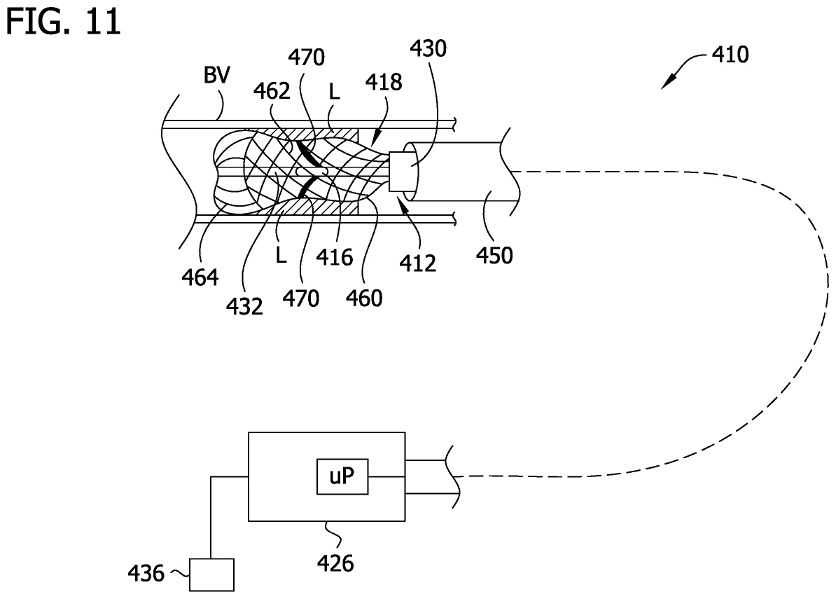

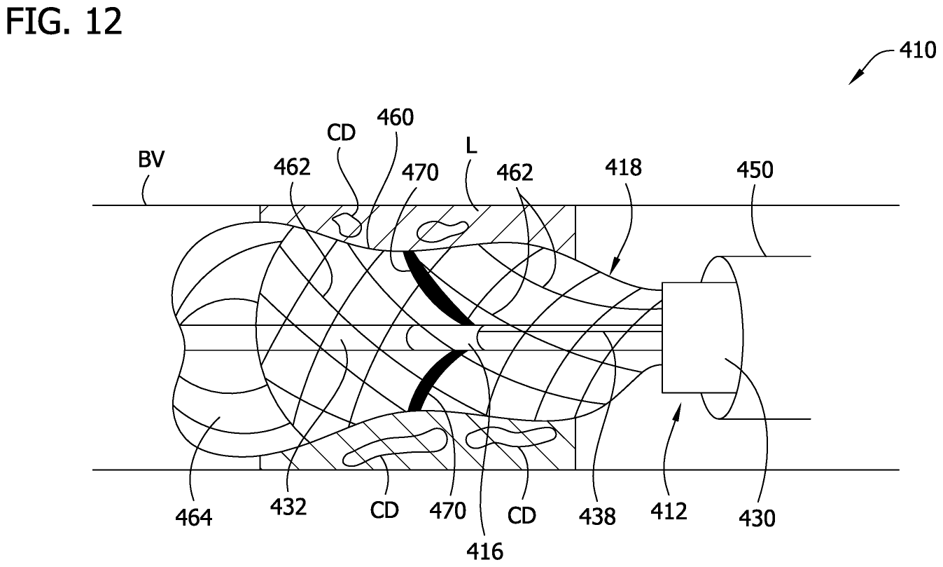

Referring to FIGS. 11 and 12, another embodiment of a treatment catheter for treating calcified plaque within a body of a subject is generally indicated at reference numeral 410. In general, the catheter 410 is configured to modify or disrupt the calcified plaque using vibrational energy. In one example, the calcified plaque may be treated further, such as through angioplasty or atherectomy or other treatments, or the treatment catheter 410 may be the primary or only treatment of the calcified plaque.

The catheter 410 includes a catheter body, generally indicated at 412, a vibration generator 416 disposed in an expandable cage, generally indicated at 418, and a control unit 426 in communication with the vibration generator. The catheter body 412 is designed and constructed to be percutaneously inserted into a blood vessel or other body lumen of the subject to deliver the expandable cage 418 to the treatment site including calcified plaque. As non-limiting examples, the catheter body 412 may have a length from about 132 cm to about 142 cm, and a diameter from about 17 mm to about 20 mm. In the illustrated embodiment, the catheter body 412 includes a retractable sheath 430 and an inner shaft 432 to which the expandable cage 418 is coupled.

The vibration generator 416 is configured to generate mechanical vibration. In one example, the vibration generator 416 comprises a piezoelectric actuator, such as a piezoelectric cylinder or tube actuator configured to generate radial vibrations. The piezoelectric actuator 416 may have an outer diameter from about 1.5 mm to about 0.5 mm, for example. A source of electrical energy 436 (e.g., a voltage source) is electrically connected to the piezoelectric actuator, such as by one or more electrical conductors 438 (FIG. 12). The electrical energy supplied to the vibration generator 416 may be controlled or operated by the control unit, which may include a microprocessor and/or a pulse width modulator. The control unit 426 may be configured to send a control signal to the piezoelectric actuator 416 to generate mechanical vibrations. The control signal delivered to the piezoelectric actuator 416 may be pulse width modulated or the parameters of the control signal may be adjusted in other ways by the control unit 426. In one example, the control unit 426 is configured (e.g., programmed) to deliver range of voltages to the piezoelectric actuator 416 to generate vibrations across a range of frequencies, for reasons explained in more detail below. The source of electrical energy 436 and/or the control unit 426 may be housed within a handle or may be separate from the handle.

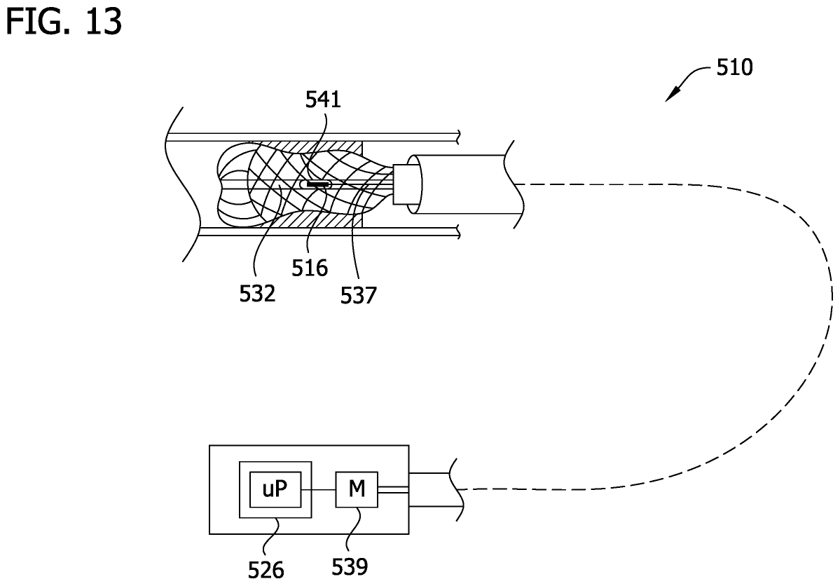

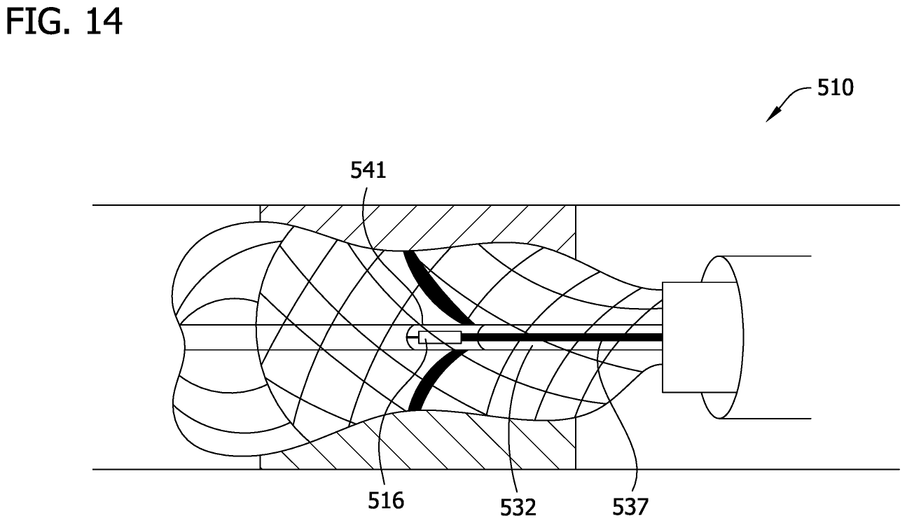

The vibration generator may comprise other types of a vibration generators suitable for generating mechanical vibrations. For example, referring to FIGS. 13 and 14, a vibration generator 516 of another catheter embodiment 510 may comprise a rotatable mass. In one example, the rotatable mass 516 is configured to generate vibrations when it reaches a certain rotational speed. In another example, the rotatable mass 516 may be an eccentric. The rotatable mass 516 may be rotated by a drive shaft 537 (e.g., a drive coil) operatively connected to a motor 539 (e.g., electrical motor) to drive rotation of the drive shaft about its axis. The motor 539 may be controlled or operated by a control unit 526, which may include a microprocessor. The control unit 526 may be configured to control a speed of the motor 539 to generate mechanical vibrations at the rotatable mass 516. In one example, the control unit 526 is configured (e.g., programmed) to control the motor 539 to generate different rotational speeds to generate vibrations across a range of frequencies, for reasons explained in more detail below. The drive shaft 537 may extend through a lumen defined by the inner shaft 532. The rotatable mass 516 is housed within and rotatable relative to a casing 541. The rotatable mass 516 may be rotatably connected to the casing 541. The motor and/or the control unit may be housing within a handle or may be separate from the handle.

For ease of disclosure, the following features are discussed only with respect to catheter 410. However, unless otherwise indicated the following disclosure applies equally to either catheter 410, 510, or any other embodiments including a type of vibration generator.

Referring back to FIGS. 11 and 12, the expandable cage 418 is configured to be received in the retractable sheath 430, which is in turn received in a guide catheter 450.

The retractable sheath 430 is retractable relative to the expandable cage 418. The expandable cage 418 comprises a cage body 460 including a plurality of struts 462 or other structural members configured to enable self-expansion of the cage when the cage is removed from the sleeve 430, such as by retracting the sleeve. As an example, the cage body 460 may generally be in the form of a self-expanding stent. The cage body 460 may comprise or be formed from a metal (e.g., Nitinol), polymer, or other material suitable for transmitting mechanical vibrations. Upon expansion, the cage 460 radially engages a calcified plaque L in the body (e.g., a blood vessel BV). The illustrated cage 460 further comprises a distal cover or cap 464 secured to the cage body 460. The distal cap 464 is configured to capture tissue that detaches from the calcified plaque L during treatment to inhibit downstream embolism. The distal cap 464 may include a blood-permeable membrane or other material suitable to capture detached tissue.

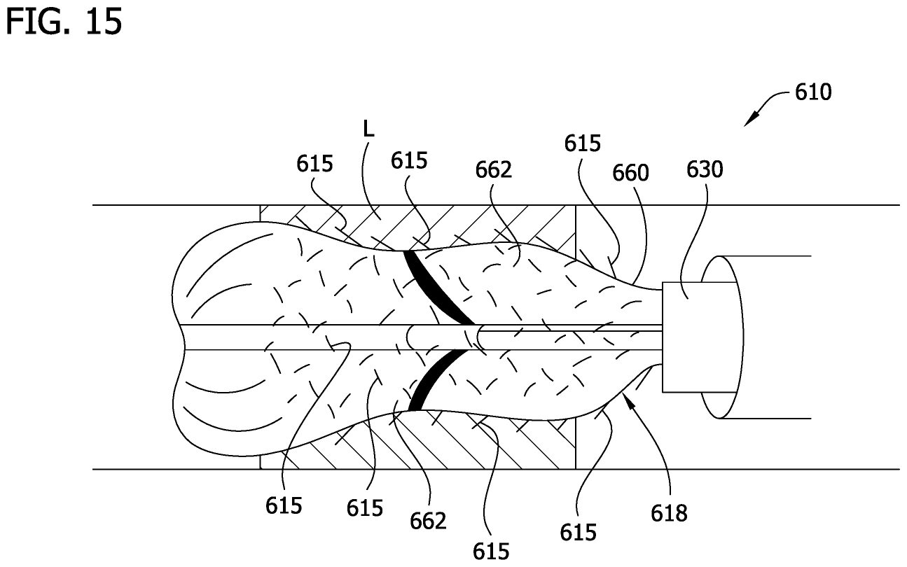

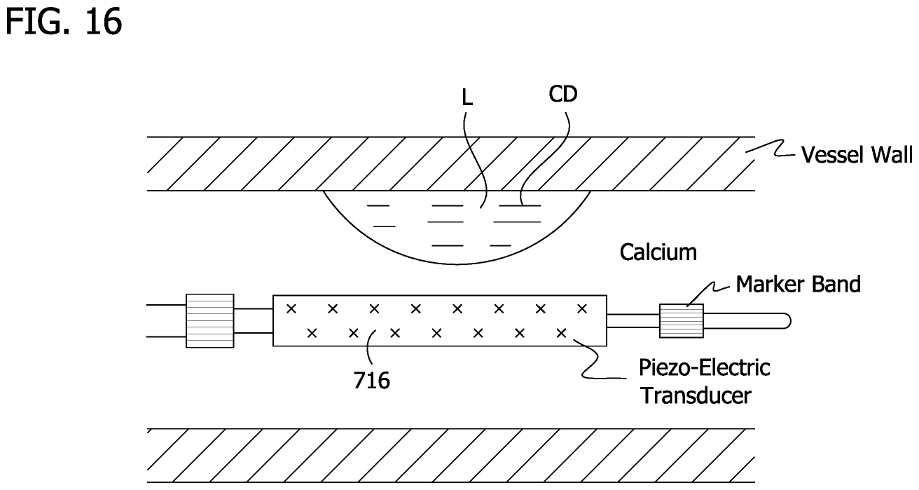

Referring to FIG. 15, the expandable cage 618 of another catheter embodiment 610 may include needles or barbs 615 (e.g., microneedles) coupled to the cage body 660 and extending generally radially outward therefrom. The needles 615 are configured to embed in the calcified plaque L upon expansion of the cage 618 and transmit the mechanical vibrations from the cage body 660 into the calcified plaque. It is believed this further facilitates transmission of vibrations into the calcified plaque L. The needles 615 may have lengths (or radial extents) of less than 1 mm (such as 0.5 mm) so that the needles do not penetrate through the wall of a blood vessel, for example, that does not have a calcified plaque. The needles 615 may be formed on or otherwise directly coupled to the cage body 660 (e.g., to the struts 662 of the cage body). In another example, the needles 615 may be formed on a mesh or sleeve that is received on the expandable cage 618. As shown in FIG. 15, the needles 615 may be angled toward the distal end of the cage body 660 when the cage 618 is expanded to inhibit damage to the needles during tracking, deployment, and recapture of the expandable cage. The needles 615 may flex or deflect toward the cage body 660 during recapture and when received in the sheath 630, and rebound away from the cage body when released from the sheath. The needles 615 may be formed from or comprise metal (e.g., stainless steel, Nitinol, titanium) or a polymer (e.g., polyimide, silicone) or other material suitable for transmitting mechanical vibrations. In one example, the needles 615 may be configured to break off from the cage body 660 upon recapture and remain in the calcified plaque L. The needles may be dissolvable or non-dissolvable.

Referring back to FIGS. 11 and 12, in the illustrated embodiments, the proximal and distal end portions of the expandable cage 418 are coupled to the inner shaft 432. In the illustrated embodiment, the inner shaft 432 defines an inner guidewire lumen configured to receive a guidewire (not shown) for delivering the catheter 410 to the treatment site. In the illustrated embodiment, the vibration generator 416 is mounted on or otherwise coupled to the inner shaft 432 within the expandable cage 418. In one example, the inner shaft 432 passes through the vibration generator 416. For example, where the vibration generator is a piezoelectric tube 416, the inner shaft 432 may pass through the tube. In other embodiments, the inner shaft 432 may be coupled to the piezoelectric tube 416 such that a lumen defined by the tube is in communication with the lumen of the inner shaft 432, thereby together defining the guidewire lumen. In another example, the rotatable mass 516 and the rotatable drive shaft 537 may include lumens to define the inner guidewire lumen through which a guidewire is received.

Referring still to FIGS. 11 and 12, the vibration generator 416 is operatively coupled to the cage 418, more specifically the cage body 460, via at least one transmission coupler 470 configured to transmit mechanical vibrations from the vibration generator 416 to the cage. (Similar transmission couplers 570 shown in FIG. 14 are coupled to the casing 541 and the cage to transmit vibrations from the rotatable mass 516.) The illustrated embodiment includes a plurality of elongate transmission couplers 470, which may be in the form of transmission struts extending generally radially outward from the vibration generator 416 (or casing) to the strut(s) 462 of the cage body 460. The transmission coupler(s) 470 may extend at a non-perpendicular angle to the cage body 460.

Reference is now made to catheter 410 for illustrated purposes with the understanding that the following disclosure applies equally to other embodiments unless otherwise indicated. In one example of use, the catheter 410 is delivered to the calcified plaque L through the guide catheter 450. For instance, the catheter 410 may be tracked along a guidewire received in the guidewire lumen of the catheter body 412. The catheter 410 may be delivered to the calcified plaque L in other ways. The retractable sheath 430 is retracted relative to the expandable cage 418 to release the expandable cage. The cage 418 self-expands as the sheath 430 is retracted, whereby the cage body radially engages the calcified plaque L. In the embodiment that includes the needles 615 (FIG. 15), the needles embed in the calcified plaque L when the cage 418 is expanded. The vibration generator 416 is then activated to generate mechanical vibrations which are transmitted to the expandable cage 418 (and needles where applicable) through the transmission coupler(s). The control unit 426 controls frequency and/or amplitude of the generated vibrations and sweeps through frequencies of vibrations to induce resonance in calcified deposits CD (FIG. 12) in the calcified plaque L. When the mechanical vibrations cause one or more calcified deposits CD to oscillate at the calcified deposit's natural frequency of vibration (its resonance frequency or resonant frequency), the calcified deposit responds at a greater amplitude of vibration. This increased amplitude fractures, disrupts, and/or modifies the calcified deposits CD in the calcified plaque L.

In one example, the vibrations generated by the vibration generator 416 (e.g., the piezoelectric actuator or the rotatable mass) may have frequencies from about 10 kHz to about 1,000 kHz and amplitude of about 10 micrometers to about 100 micrometers. The resonant frequency of hydroxyapatite (a primary material in calcified deposits) is 100-280 kHz. Accordingly, in one example the catheter 410 is configured to transmit mechanical vibrations across this frequency range (i.e., sweep through this frequency range) to induce resonance and break up the calcified deposits. The catheter 410 may be configured to transmit other frequency ranges. The frequencies are generated using the control unit 426.

After treatment, the expandable cage 418 collapses as it is retracted back into the retractable sheath 430, or alternatively, as the sheath is moved distally to recapture the cage. As the cage 418 collapses, tissue that detached from the calcified plaque L enters the expandable cage and is captured in the distal cap 464. The catheter 410 is then withdrawn from the body. Subsequent treatment (e.g., angioplasty and/or atherectomy and/or drug treatment) may then be performed.

Referring to FIG. 15, in another embodiment a catheter 710 does not include the expandable cage or other transmission component. Instead, the vibration generator 716 is configured to transmit the vibrations through body fluid (e.g., blood) in the body (e.g., blood vessel). The vibration generator 716 may be delivered to the calcified plaque L so that the generator is radially spaced (e.g., about 1 mm) from the calcified plaque L. Thus, the body fluid acts at a medium transmitting the mechanical vibrations to the calcified plaque L to induce resonance of the calcified deposits CD. The vibration generator 716 may be a piezoelectric tube, for example, or other vibration generator. Other than this difference, the catheter operates similar to the prior embodiment in that the catheter delivers vibrations to produce resonance of the calcified deposits in the calcified plaque to break up, fracture, or otherwise modify the calcified plaque.

Several benefits are realized by the use of the vibration generator to induce resonance of calcified deposits in a calcified plaque. For example, the catheter may prepare the calcified plaque for subsequent interventions by disrupting, modifying, and/or removing calcified deposits from the calcified plaque. The catheter may modify and remove calcified deposits using a single device. The catheter may be compatible with a 0.014 in guidewire and a 6F guide catheter. There is no occlusion of the body lumen during treatment when the expandable cage comprises struts, and therefore, openings.

Moreover, there is no damage to healthy regions of the body lumen using the catheter.

The invention may be further described by reference to the following numbered paragraphs:

1. A catheter for treating calcified plaque within a body of a subject, the catheter comprising:
a catheter body having opposite proximal and distal end portion and a longitudinal axis extending therebetween, wherein the catheter body is configured to be percutaneously inserted into the body of the subject and delivered to a treatment site having calcified plaque;
an expandable balloon coupled to the distal end portion of the catheter body, wherein the expandable balloon is configured to contact the calcified plaque and apply a radial pressure thereto;
a thermal shock generator operatively coupled to the expandable balloon and configured to alternate between heating and cooling the calcified plaque to induce thermal shock in the calcified plaque.

2. The catheter set forth in paragraph 1, wherein a wall of the expandable balloon is thermally conductive to transfer heat between the calcified plaque and the expandable balloon.

3. The catheter set forth in paragraph 2, wherein the thermal shock generator includes a heating system configured to deliver heated thermally conductive fluid to the balloon, and a cooling system configured to deliver cooled thermally conductive fluid to the balloon.

4. The catheter set forth in paragraph 3, wherein the heating system includes a heating circulator for circulating the heated thermally conductive fluid into and out of the balloon, wherein the cooling system includes a cooling circulator for circulating the cooled thermally conductive fluid into and out of the balloon.

5. The catheter set forth in paragraph 3, further comprising a control unit in communication with the heating system and the cooling system, wherein the control unit is configured to alternate between operating the heating system and operating the cooling system.

6. The catheter set forth in paragraph 5, further comprising a temperature sensor configured to sense at least one of a temperature inside the balloon, a temperature of the balloon wall, and a temperature of the calcified plaque, wherein the temperature sensor is in communication with the control unit.

7. The catheter set forth in paragraph 2, wherein the thermal shock generator is configured to heat the wall of the balloon to a temperature from about 150 C to about 300 C, and cool the balloon wall at a rate of from about −20 C/s to about−40 C/s.

8. The catheter set forth in paragraph 8, wherein the thermal shock generator is configured to cool the balloon wall to a temperature from about −38 C to about−40 C.

9. The catheter set forth in paragraph 1, wherein the thermal shock generator includes a plaque heating element configured to non-conductively heat the calcified plaque.

10. The catheter set forth in paragraph 9, wherein the plaque heating element comprises an ultrasonic transducer.

11. The catheter set forth in paragraph 9, wherein the plaque heating element comprises a radiofrequency generator.

12. The catheter set forth in paragraph 9, wherein the plaque heating element is disposed in the balloon.

13. The catheter set forth in paragraph 9, wherein the thermal shock generator is configured to deliver refrigerant to the balloon to cool the balloon and the calcified plaque.

14. The catheter set forth in paragraph 1, wherein the thermal shock generator is configured to deliver refrigerant to the balloon to cool the balloon and the calcified plaque.

15. A method of treating calcified plaque at a treatment site within a body of a subject, the method comprising:
delivering a catheter body of a catheter to the treatment site so that a balloon at a distal end portion of the catheter body is adjacent the calcified plaque;
expanding the balloon after said delivering the catheter body to apply radial pressure to the calcified plaque;
heating the calcified plaque; and
rapidly cooling the heated calcified plaque to induce thermal shock in the calcified plaque simultaneously with the radial pressure applied to the calcified plaque by the expandable balloon.

16. The method set forth in paragraph 15, wherein said heating the calcified plaque comprises delivering heated thermally conductive fluid into the balloon.

17. The method set forth in paragraph 15, wherein said cooling the calcified plaque comprises delivering cooled thermally conductive fluid into the balloon.

18. The method set forth in paragraph 15, wherein said heating the calcified plaque comprises non-conductively heating the calcified plaque using a plaque heating element coupled to the catheter body.

19. The method set forth in paragraph 15, wherein said cooling the calcified plaque comprises introducing refrigerant into the balloon to cool the balloon.

20. A catheter for treating calcified plaque within a body of a subject, the catheter comprising:
a catheter body having opposite proximal and distal end portion and a longitudinal axis extending therebetween, wherein the catheter body is configured to be percutaneously inserted into the body of the subject and delivered to a treatment site having calcified plaque;
an expandable balloon coupled to the distal end portion of the catheter body, wherein the expandable balloon is configured to contact the calcified plaque and apply a radial pressure thereto;
a nuclear magnetic resonance generator including a radiofrequency coil within the balloon, wherein the nuclear magnetic resonance generator is configured to disrupt the calcified plaque.

21. A catheter for treating calcified plaque within a body of a subject, the catheter comprising:
a catheter body having opposite proximal and distal end portion and a longitudinal axis extending therebetween, wherein the catheter body is configured to be percutaneously inserted into the body of the subject and delivered to a treatment site having calcified plaque; and
a vibration generator at the distal end portion of the catheter body, the vibration generator configured to generate radial mechanical vibrations suitable to produce resonance in calcified deposits in the calcified plaque, thereby disrupting the calcified deposits.

22. The catheter set forth in paragraph 21, further comprising an expandable cage at the distal end portion of the catheter body, the expandable cage configured to be expandable to radially engage the calcified plaque, wherein the vibration generator is operatively coupled to the expandable cage so that the mechanical vibrations generated by vibration generator are transmitted to the expandable cage and in turn transmitted to the calcified plaque.

23. The catheter set forth in paragraph 22, wherein the vibration generator is disposed in the expandable cage.

24. The catheter set forth in paragraph 23, wherein the vibration generator is operatively coupled to the expandable cage by at least one transmission coupler.

25. The catheter set forth in paragraph 21, wherein the vibration generator comprises a piezoelectric actuator.

26. The catheter set forth in paragraph 25, wherein the vibration generator comprises a piezoelectric tube.

27. The catheter set forth in paragraph 22, wherein the expandable cage comprises a cage body including a plurality of struts.

28. The catheter set forth in paragraph 22, wherein the expandable cage comprises a plurality of needles configured to embed in the calcified plaque.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1-15. (canceled)

16. A catheter for treating calcified plaque within a body of a subject, the catheter comprising:
a catheter body having opposite proximal and distal end portions and a longitudinal axis extending therebetween, wherein the catheter body is configured to be percutaneously inserted into the body of the subject and delivered to a treatment site having calcified plaque;
an expandable balloon coupled to the distal end portion of the catheter body, wherein the expandable balloon is configured to contact the calcified plaque and apply a radial pressure thereto; and
a thermal shock generator operatively coupled to the expandable balloon and configured to alternate between heating and cooling the calcified plaque to induce thermal shock in the calcified plaque.

17. The catheter set forth in claim 16, wherein a wall of the expandable balloon is thermally conductive to transfer heat between the calcified plaque and the expandable balloon.

18. The catheter set forth in claim 17, wherein the thermal shock generator includes a heating system configured to deliver heated thermally conductive fluid to the expandable balloon, and a cooling system configured to deliver cooled thermally conductive fluid to the expandable balloon.

19. The catheter set forth in claim 18, wherein the heating system includes a heating circulator for circulating the heated thermally conductive fluid into and out of the expandable balloon, wherein the cooling system includes a cooling circulator for circulating the cooled thermally conductive fluid into and out of the expandable balloon.

20. The catheter set forth in claim 18, further comprising a control unit in communication with the heating system and the cooling system, wherein the control unit is configured to alternate between operating the heating system to deliver heated thermally conductive fluid to the expandable balloon, and operating the cooling system to deliver cooled thermally conductive fluid to the expandable balloon.

21. The catheter set forth in claim 20, further comprising a temperature sensor configured to sense at least one of a temperature inside the expandable balloon, a temperature of the wall of the expandable balloon, and a temperature of the calcified plaque, wherein the temperature sensor is in communication with the control unit.

22. The catheter set forth in claim 17, wherein the thermal shock generator is configured to heat the wall of the expandable balloon to a temperature from about 150 C to about 300 C, and cool the wall of the expandable balloon at a rate of from about −20 C/s to about −40 C/s.

23. The catheter set forth in claim 22, wherein the thermal shock generator is configured to cool the wall of the expandable balloon to a temperature from about −38 C to about −40 C.

24. The catheter set forth in claim 16, wherein the thermal shock generator includes a plaque heating element configured to non-conductively heat the calcified plaque.

25. The catheter set forth in claim 24, wherein the plaque heating element is disposed in the expandable balloon.

26. The catheter set forth in claim 16, wherein the thermal shock generator is configured to deliver refrigerant to the balloon to cool the balloon and the calcified plaque.

27. A method of treating calcified plaque at a treatment site within a body of a subject using the catheter set forth in claim 16, the method comprising:
delivering the catheter body to the treatment site so that the expandable balloon is adjacent the calcified plaque;
expanding the expandable balloon, after said delivering the catheter body, to apply radial pressure to the calcified plaque;
heating the calcified plaque using the thermal shock generator; and
rapidly cooling the heated calcified plaque using the thermal shock generator to induce thermal shock in the calcified plaque simultaneously with the radial pressure applied to the calcified plaque by the expandable balloon.

28. A catheter for treating calcified plaque within a body of a subject, the catheter comprising:
a catheter body having opposite proximal and distal end portions and a longitudinal axis extending therebetween, wherein the catheter body is configured to be percutaneously inserted into the body of the subject and delivered to a treatment site having calcified plaque; and
a nuclear magnetic resonance generator including a radiofrequency coil coupled to a distal end portion of the catheter body, wherein the nuclear magnetic resonance generator is configured to disrupt the calcified plaque.

29. A catheter for treating calcified plaque within a body of a subject, the catheter comprising:
a catheter body having opposite proximal and distal end portions and a longitudinal axis extending therebetween, wherein the catheter body is configured to be percutaneously inserted into the body of the subject and delivered to a treatment site having calcified plaque; and
a vibration generator at the distal end portion of the catheter body, the vibration generator configured to generate radial mechanical vibrations suitable to produce resonance in calcified deposits in the calcified plaque, thereby disrupting the calcified deposits.

30. The catheter set forth in claim 29, further comprising an expandable cage at the distal end portion of the catheter body, the expandable cage configured to be expandable to radially engage the calcified plaque, wherein the vibration generator is operatively coupled to the expandable cage so that the mechanical vibrations generated by vibration generator are transmitted to the expandable cage and in turn transmitted to the calcified plaque.

31. The catheter set forth in claim 30, wherein the vibration generator is disposed in the expandable cage.

32. The catheter set forth in claim 31, wherein the vibration generator is operatively coupled to the expandable cage by at least one transmission coupler.

33. The catheter set forth in claim 29, wherein the vibration generator comprises a piezoelectric actuator.

34. The catheter set forth in claim 30, wherein the expandable cage comprises a cage body including a plurality of struts.

35. The catheter set forth in claim 30, wherein the expandable cage comprises a plurality of needles configured to embed in the calcified plaque.
